(19) United States
(12) Patent Application Publication
(10) Pub. No.: US 2026/0137599 A1
(43) Pub. Date: May 21, 2026

(54) ENTERAL FEEDING PUMP WITH EXPANDED FUNCTIONALITY

(71) Applicants: Florida State University Research Foundation, Inc., Tallahassee, FL (US); Adjuvo Healthworks LLC, Panama City, FL (US)

(72) Inventors: Heather Zawahry, Panama City, FL (US); Yvonne Marie Traynham, Panama City, FL (US); Mauricio Chagas, Panama City, FL (US)

(21) Appl. No.: 19/347,870

(22) Filed: Oct. 2, 2025

Related U.S. Application Data
(60) Provisional application No. 63/703,361, filed on Oct. 4, 2024.

(51) Int. Cl. A61J 15/00; A61M 5/168

(52) U.S. Cl. CPC A61J 15/0076; A61M 5/16804; A61M 2205/502

(57) ABSTRACT

In accordance with the purpose(s) of the present disclosure, as embodied and broadly described herein, the disclosure relates to enteral feeding pumps with expanded functionality, enteral feeding pump systems, methods of operating enteral feeding pump systems, and the like.

### CROSS-REFERENCE TO RELATED APPLICATIONS

This application claims the benefit of and priority to co-pending U.S. Provisional Ser. No. 63/703,361 , filed on Oct. 4, 2024, the contents of which are incorporated by reference herein in its entirety.

### BACKGROUND

Mother's own milk (MOM) can be important for neonates, particularly those in the neonatal intensive care unit (NICU), due to its numerous health benefits, which can contribute significantly to improved outcomes for premature and critically ill infants. Further, positive sensory experiences for these infants can activate neural pathways and contribute to the structural maturation of their brains. Being at mother's breast can provide the optimal tactile, visual, auditory, and olfactory stimulation. In order to add the gustatory component, NICU nurses and therapists often introduce drops of MOM administered with a syringe into the infant's mouth as she explores mom's breast. The smallest, most vulnerable infants often only tolerate a few microliters of liquid introduced orally at a time. This process can be cumbersome and time consuming for NICU personnel.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 shows a schematic of an enteral feeding pump with expanded functionality according to various embodiments of the present disclosure.

FIG. 2 shows a schematic of an example process flow of an enteral feeding pump with expanded functionality according to various embodiments of the present disclosure.

FIG. 3 shows a drawing of a network environment according to various embodiments of the present disclosure.

FIG. 4 shows a flowchart illustrating one example of functionality implemented as a portion of an application executed in a computing device in the network environment of FIG. 3 according to various embodiments of the present disclosure.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed devices, systems, and methods belong. It will be

further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a fluid” include, but are not limited to, mixtures or combinations of two or more such fluids, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x′ and less than ‘y'. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to human and constituents thereof.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

It is understood that the components disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

As used herein, a “fluid” can refer to any flowable substance intended for delivery through an enteral feeding pump system, including liquid nutritional formulations, hydration solutions, medication suspensions, breast milk, baby formula, or other enteral or parenteral compositions. A fluid can be Newtonian or Non-Newtonian, including fluids with varying viscosities. A fluid can include a homogenous or heterogenous mixture, emulsion, or suspension suitable for therapeutic or nutritional administration to a patient.

As used herein, a “housing” can refer to a structural enclosure or casing that supports, contains, and/or protects one or more internal components of an enteral feeding pump system. A housing can contain one or more motors, a power supply, control electronics, fluid delivery system components, one or more sensors, one or more motor drivers, various user interface elements, etc. A housing can be constructed from any suitable material (e.g., polymers, metals, composites, etc.) and can be designed as a single unit or multiple interconnected parts. For example, a housing can be constructed from polycarbonate, polypropylene, acrylonitrile butadiene styrene, polyamide, high-density polyethylene, thermoplastic elastomers, silicone rubber, stainless steel, etc. A housing can include external features such as mounting elements, plug ports, a display, one or more buttons, etc.

As used herein, a “plug port” can refer to a receptacle on the housing or associated component configured to receive a corresponding plug or electronic connector. A plug port can facilitate mechanical and/or electrical connection between an enteral feeding pump device and an external device (e.g., a client device), an accessory (e.g., a remote control), or a power source. A plug port can include an electrical power input port, data communication interfaces (e.g., universal serial bus (USB)), sensor inputs, or electronic connectors. A plug port can include features such as alignment guides, sealing elements, locking mechanisms, or protective covers to ensure secure, hygienic, and reliable operation.

As used herein, an “actuator” can refer to a mechanical component or assembly configured to convert energy (e.g., rotational or linear motion) into a controlled mechanical movement that engages with and drives a fluid reservoir to initiate, regulate, or terminate fluid flow. An actuator can be driven by a motor or other energy source, and can produce linear, rotational, or oscillatory motion. An actuator can operate in response to a user input or instruction and can include various elements such as levers, shafts, screws, sliders, plungers, gear trains, and the like. In some examples, an actuator can include various components such as gears, screws, shafts, linkages, and the like configured to convert a motor's rotational motion into a linear form of actuation used to control the position, speed, or force of a connected mechanical element or system that drives a fluid reservoir to initiate, regulate, or terminate fluid flow. For example, an actuator can be configured to convert a motor's rotational motion into a linear form of actuation used to control the position, speed, or force of a clamp connected to a plunger of a syringe.

As used herein, a “bay” can refer to a defined structural compartment, recess, or cavity within a housing that can be configured to receive, retain, or interface with a component, module, or accessory of an enteral feeding pump system. A bay can be designed to accommodate one or more fluid reservoirs, one or more tubes, a power supply unit, a battery pack, one or more actuators, one or more motors, and/or associated motor drivers. In some examples, a bay can be designed to accommodate one or more air supply canisters in fluidic communication with one or more purge fittings of a tube. A bay can include alignment features, electrical or mechanical connectors, locking mechanisms, or sensor interfaces to ensure proper positioning and functional engagement of an inserted component. A bay can be open, partially enclosed, or fully enclosed and in some examples can be accessible to a user through a hinged access door within the housing. In some examples, a bay can include a clamp assembly configured to engage a flange of a syringe or other structural component of a fluid reservoir.

As used herein, a “motor” can refer to an electromechanical device configured to convert electrical energy into mechanical motion for the purpose of driving one or more components of an enteral feeding pump system. A motor can generate rotational or linear output and can be operatively coupled to mechanical elements such as actuators, gears, or drive shafts to initiate, regulate, or terminate fluid flow. A motor can include a brushed or brushless DC motor, a stepper motor, a servo motor, or other type of compatible motor. A motor can be controlled by a motor driver in data communication with a computing device and can include feedback mechanisms such as one or more sensors to enable a closed-loop operation.

As used herein, a “fluid reservoir” can refer to a self-contained, typically disposable or replaceable unit that holds or conveys a fluid and is configured to be operatively received by a corresponding bay or interface of an enteral feeding pump system. A fluid reservoir can store a liquid nutritional formulation, medication, hydration solution, or other fluid, and can further operatively connect to a tube, a connector of a tube, and/or an actuator to facilitate fluid delivery to a patient. A fluid reservoir can be constructed from medical-grade materials suitable for single or limited use and can be pre-filled or fillable by a user. In various examples, a fluid reservoir can be constructed from polypropylene, polyethylene, polycarbonate, silicone rubber, thermoplastic elastomers, polyvinyl chloride, or any combination thereof. In some examples, a fluid reservoir can include one or more graduation marks on an external surface to indicate a volume of fluid within the fluid reservoir.

As used herein, a “connector” can refer to a standardized interface component designed in accordance with applicable International Organization for Standardization (ISO) specifications to facilitate secure, compatible, and adjustable connection between a tube and a fluid reservoir. A connector can include male or female mating features and/or locking mechanisms. A connector can be configured to ensure leak-free, secure attachment to the corresponding tube or fluid reservoir and can be integrated into or affixed to a fluid reservoir, a tube, or both. For example, a connector could include an American National Standards Institute (ANSI)/Association for the Advancement of Medical Instrumentation (AAMI)/International Organization for Standardization (ISO) 80369-3 compliant twist lock connector.

As used herein, a “tube” can refer to a flexible or semi-rigid hollow cylindrical conduit configured to convey a fluid between a fluid reservoir and a patient. A tube can be constructed from biocompatible, medical-grade materials (e.g., silicone, polyurethane, polyethylene, polyvinyl chloride, etc.). A tube can vary in length, diameter, wall thickness, and can include integrated connectors to facilitate secure attachment to other components and/or a patient. In some examples, a tube can be configured to convey a fluid between a fluid reservoir and a mother's breast.

As used herein, a “purge fitting” or “purge port” can refer to a component or device configured to remove air from a tube before or during fluid delivery to a patient. A purge fitting can include a valve, a port for a syringe, and/or one or more clamps configured to isolate and push air bubbles out of a section of a tube. In some examples, a purge fitting can be in fluidic communication with an air supply. A purge fitting can be utilized within an enteral feeding pump system to ensure accurate fluid delivery.

### Enteral Feeding Pumps, Enteral Feeding Pump Systems, and Methods of Making and Using the Same

Existing enteral feeding pumps can lack the flexibility of function to deliver a very small amount of fluid in a discrete aliquot. Further, they often lack a manual mode which could allow NICU personnel and properly trained parents to administer aliquots as appropriate. As NICU infants mature, they can acquire the ability to tolerate drops of oral nutrition administered automatically, at regular intervals. Further, NICU feeding specialists can desire to administer nutrition in a physiologic manner, with predetermined small aliquots of nutrition introduced orally followed sequentially by larger aliquots introduced directly to the infant's stomach via a gavage feeding tube. Existing enteral feeding pumps often lack this functionality.

In one aspect, the enteral feeding pumps with expanded functionality described herein can improve or enhance the administration of oral nutrition in a subject. In various aspects, the enteral feeding pumps described herein could provide nutrition in an intermittent mode via droplets or in a continuous mode at a prescribed rate utilizing a programmable control system, motorized independent actuation to individual supply reservoirs (e.g., fluid reservoirs), and a pre-sized tubing delivery assembly for known volume and rate control. The pre-packaged tubing delivery system could have a purge port and constant tube length geometry. In various aspects, the pre-packaged tubing delivery system can be supplied as a disposable sterile assembly. Pre-programmed routines based on the total volume of the fluid reservoir and delivery system can activate positive displacement linear actuation at a controlled rate. In some aspects, there can be a data acquisition capability to allow for export to the patient medical record. A purge port at the tip of the delivery system can be utilized to supply air through the tubing for purging fluid. Further, in some aspects, remote operation via a plug-in cord with a switch or wireless remote control can allow a nursing mother to have local control to stop, start, or pause a supply, or deliver one or more aliquots of a fluid. The controller can be clipped to the garment of the nursing mother to facilitate placement for the most natural position of the infant and mother. A healthcare professional could also have local control at the unit.

In another aspect, the unit can have modality for various functions, including: I. Off, II. Fill, III. Continuous, IV. Intermittent, V. Alarm, VI. Purge, and VII. Pause. The modes can function by either automatic or manual operation. The fluid reservoir (e.g., a syringe) could be placed vertically in a “supply bay” which could have a support that serves a range of standard diameter sizes for placement at a designated height centered above an actuator. The pre-sized tip and tubing delivery assembly could be attached to the tip of the reservoir syringe. On operation, a motor (e.g., a stepper motor) can force the actuator to move upward in positive displacement for controlled delivery to the infant patient. The unit can be pre-programmed based at least in part on the known volume of the individual fluid reservoir and delivery system at a designated rate. The controller logic could operate each actuator individually, or a plurality of actuators could be operated as a single actuation, simultaneously, or sequentially. Simultaneous operation could allow oral and gavage feeding, respectively, to stimulate the infant via the oral component while providing sufficient nutrition via the gavage tube feeding.

### Housing

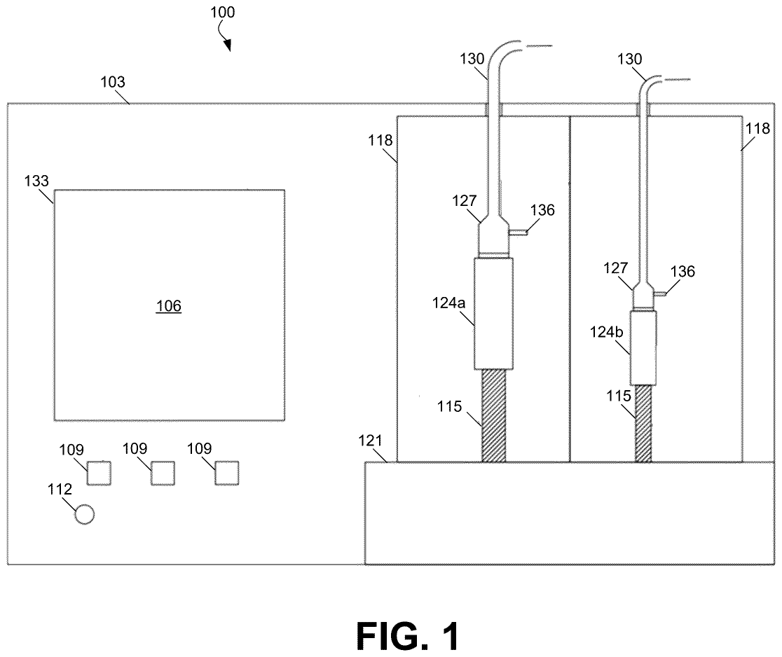

In various aspects, the enteral feeding pump of the present disclosure can include a housing for component mounting, protection, and structural support. Referring to the example of FIG. 1, an enteral feeding pump device 100 can include a housing 103, where one side of the housing 103 can be dedicated to device monitoring and control. The housing 103 can provide structure to support a user interface 106 (e.g., a flush mounted screen), manual push buttons 109, and remote operation plug-in access through one or more plug ports 112. Internally, the housing 103 can include one or more computing devices (e.g., a microcontroller), motor drivers, sensors, and internal wiring.

In the example of FIG. 1, another side of the housing 103 can be dedicated to the mechanical fluid delivery system. The housing 103 can protect the actuation assemblies 115 and other fluid delivery components. In the example of FIG. 1, two bays 118 are shown, each with a dedicated fluid delivery system. In FIG. 1, the housing 103 can serve as support for the actuation assemblies 115 (also referred to herein as actuators 115), one or more motors 121, and fluid delivery system. In some aspects, the housing 103 can have a hinged access door that can be

opened to allow insertion and securing of fluid (e.g., breast milk, formula, etc.) reservoirs 124 (e.g., delivery syringes), connectors 127, and tubes 130. The access door can then be closed during operation. A side of the housing 103 can include openings to support and guide the fluid tubes 130 from the device 100.

### Programmable Computing Device(s)

One or more computing devices, such as a microcontroller, can be pre-programmed with routines for the method of operation. Data acquisition capability can also be built in for documentation of fluid volume delivery or fluid delivery rate. A user interface 106 (e.g., a screen, a computer, a tablet, a phone, one or more manual push buttons 109, etc.) can be connected for programming and data acquisition purposes. While the example device 100 of FIG. 1 shows a user interface 106 mounted on the housing 103 of the device 100, in various aspects a user interface 106 can be external to the housing 103 (e.g., a tablet, computer, remote operation accessory, and/or phone in wired or wireless communication with the computing device of the device 100).

### Display

In some aspects, the device 100 can include a display 133 (e.g., a screen) that can render the user interface 106 to indicate the programmed routine and/or receive a user input.

### Manual Push Buttons

Manual push buttons 109 can be used for local control and can include a start button, a stop button, an aliquot button configured to deliver a predetermined aliquot of fluid, or a pause button for operation by a user, such as a health care professional.

### Switch to Power Supply

A power switch can be used to turn on and off a power supply to the device 100. In various aspects, the power supply can have a 120V single phase input transformed to a control voltage for the computing devices, motor drivers, sensors, and/or motors.

### External Control Assembly/Remote Operation

In various aspects, the device 100 can include one or more plug ports 112 to establish data communication with a remote operation accessory having push button control to allow a user to remotely stop the operation of the device 100, start the operation of the device 100, pause the operation of the device 100, deliver one or more aliquots of fluid, or end the operation of the device 100.

In various aspects, the device 100 can be configured to wirelessly communicate with a remote operation accessory having push button control to allow a user to remotely stop the operation of the device 100, start the operation of the device 100, pause the operation of the device 100, deliver one or more aliquots of fluid, or end the operation of the device 100.

### Motor

Actuation of the fluid delivery system can be via control of one or more motors 121 providing linear and/or rotational actuation of one or more actuation assemblies 115. In various aspects, a plurality of motors 121 can allow setup for a varied size of fluid reservoirs 124 and routine. For example, as shown in FIG. 1, the device 100 could include 1) a smaller reservoir 124b and 2) a larger reservoir 124a for either intermittent or continuous delivery of fluid. These functions can be independent and can be operated individually, simultaneously, or sequentially.

### Motor Driver

Each motor 121 can have a motor driver that interfaces with the computing device to drive the motor assembly.

### Clamp Assembly

In some aspects, the device 100 can include a structure that clamps the fluid delivery system (e.g., fluid reservoir) to an actuation assembly 115 and/or a surface of a bay 118. For example, the clamping structure can hold the fluid reservoir 124 (e.g., a flange of a syringe), provide structural stability, and allow motorized actuation of the actuation assembly 115. In some examples, a clamp assembly or clamp can refer to a mechanical component configured to hold, secure, or restrain a portion, surface, or component of a fluid reservoir in a fixed position relative to another portion, component, or surface of the fluid reservoir. In various examples, a clamp can include at least two opposing surfaces or jaws that are moved toward one another to apply a holding force to the object(s) positioned between them.

### Fluid Reservoir

In various aspects, a fluid reservoir 124 can include an externally supplied syringe (a standard syringe assembly with a graduated reservoir and plunger actuation), connectors 127, and a tube 130.

In some aspects, the fluid delivery assembly can include a standard plastic medical syringe having a flanged barrel with indicated volumetric graduation lines for the fluid reservoir 124, and a push plunger to dispense the liquid through the syringe nozzle when activated by the actuation assembly 115.

The syringe nozzle can be connected to standard sized (e.g., French diameter) tubes 130 via a connector 127 (e.g., ANSI/AAMI/ISO 80369-3 compliant twist lock connectors). In some aspects, a purge port 136 can be attached in line. In various aspects, a connected feeding tube 130 can be a standard length and a pre-programed volumetric supply can be based at least in part on this known length and the internal diameter of the tube 130 for volumetric supply. In various examples, a stepper motor 121 can have a precise and accurate ability to deliver a fluid supply to a patient in an intermittent and/or continuous flow mode.

### Enteral Feeding Pump

The present disclosure provides for an enteral feeding pump device 100, including at least one motor driver, a fluid delivery system, and at least one tube 130 in fluidic communication with the fluid delivery system. The tube 130 can be configured to deliver a fluid from the fluid delivery system to a patient or a mother's breast when an actuator 115 is actuated by a motor 121. In various aspects, the fluid delivery system can include at least one motor 121 configured to actuate at least one actuator 115, where the motor(s) 121 is in data communication with at least one motor driver.

In some aspects, the fluid delivery system can further include at least one actuator 115 configured to actuate a fluid within at least one fluid reservoir 124. A fluid reservoir 124 can be configured to contain the fluid and can be in fluidic communication with at least one tube 130. Further, in some aspects, the fluid reservoir 124 can be connected to an actuator 115 at a first end and have a receiving end at a second end configured to removably connect to a tube 130. In various aspects, a tube 130 can be in fluidic communication with a fluid reservoir 124, where the tube 130 removably connects at a first end to the receiving end of the fluid reservoir 124.

In some aspects, the enteral feeding pump device 100 can include a housing 103 configured to enclose at least one motor driver and the fluid delivery system. In addition, the housing 103 can include at least one opening extending from an interior of the housing 103 to the outer surface of the housing 103, where the opening can be configured to fit a tube 130. In various aspects, the housing 103 can further include a door configured to allow an operator of the enteral feeding pump device 100 to access the fluid delivery system.

In various aspects, a tube 130 can include a purge port 136. In some aspects, the enteral feeding pump device 100 can include one or more computing devices including a processor and a memory and machine-readable instructions that, when executed by the processor, cause the one or more computing devices to at least acquire a user input, generate an instruction for at least one motor driver based at least in part on the user input, and send the instruction to at least one motor driver.

In some aspects, at least one fluid reservoir 124 can include a syringe having an internal chamber and a plunger disposed within the internal chamber of the syringe and configured to slide along an inner diameter of the internal chamber when a pressure is applied to the plunger by an actuator 115. In some aspects, the enteral feeding pump device 100 can include a user interface 106 disposed within the outer surface of the housing 103 and in data communication with one or more computing devices. In various aspects, the user interface 106 can be configured to receive a user input and at least one motor driver controls at least one motor 121 based at least in part on the user input.

### Enteral Feeding Pump System

The present disclosure also provides an enteral feeding pump system including a housing 103, a control system disposed within the interior of the housing 103, a user interface 106 disposed within an outer surface of the housing 103 and in data communication with the control system, a fluid delivery system disposed within the interior of the housing 103, and two or more tubes 130 in fluidic communication with the fluid delivery system configured to deliver a fluid from a fluid reservoir 124. In various aspects, the housing 103 can include two or more openings extending from an interior of the housing 103 to the outer surface of the housing 103, where each of the two or more openings is configured to fit a tube 130.

In various aspects, the control system can include two or more motor drivers, a computing device having a processor and a memory, and machine-readable instructions that, when executed by the processor, cause the computing device to at least acquire a user input, generate an instruction for at least one of the two or more motor drivers based at least in part on the user input, and control at least one of the two or more motor drivers based at least in part on the instruction. In various aspects, the user interface 106 can be disposed within the outer surface of the housing 103 and in data communication with the control system. In various aspects, a fluid delivery system can include two or more bays 118, where each bay 118 can have a motor 121 configured to actuate an actuator 115, where the motor 121 is in data communication with at least one motor driver. The actuator 115 can be configured to actuate a fluid within a fluid reservoir 124, and the fluid reservoir 124 can be configured to contain the fluid and in fluidic communication with the tube 130. The fluid reservoir 124 can be connected to the actuator 115 at a first end of the fluid reservoir 124 and have a receiving end at a second end of the fluid reservoir 124 configured to removably connect to the tube 130.

In various aspects, each of the two or more tubes 130 can be in fluidic communication with the fluid reservoir 124, where each of the two or more tubes 130 removably connect at a first end of the tube 130 to the receiving end of the fluid reservoir 124. Each of the two or more tubes 130 can be configured to deliver the fluid from the fluid reservoir 124 when the actuator 115 is actuated by the motor 121.

In some aspects, a tube 130 can include a purge port 136. In various aspects, the machine-readable instructions that, when executed by the processor, cause the computing device to acquire the user input can further cause the computing device to at least acquire fluid delivery data and generate the instruction for at least one of the two or more motor drivers based at least in part on the user input and the fluid delivery data.

In some aspects, the fluid reservoir 124 can include a syringe having an internal chamber and a plunger disposed within the internal chamber of the syringe and configured to slide along an inner diameter of the internal chamber when a pressure is applied to the plunger by the actuator 115. In some aspects, the enteral feeding pump system can include a first fluid reservoir 124 and a second fluid reservoir 124, where a volume of the first fluid reservoir 124 can be less than a volume of the second fluid reservoir 124. In other aspects, a volume of the first fluid reservoir 124 can be the same as a volume of the second fluid reservoir 124. In some aspects, each of the two or more bays 118 can further include a clamp assembly configured to receive a flange of the syringe.

### Method of Operating Enteral Feeding Pump

The present disclosure further provides a method of operating an enteral feeding pump device 100 including acquiring a user input, generating an instruction for at least one motor driver based at least in part on the user input, controlling, by the at least one motor driver, a motor 121 in data communication with the at least one motor driver based at least in part on the instruction, and actuating, by the motor 121, an actuator 115 configured to actuate a fluid within a fluid reservoir 124 to deliver a fluid from the fluid reservoir 124 to a tube 130. In some aspects, acquiring the user input can further include acquiring fluid delivery data and generating the instruction for at least one motor driver based at least in part on the user input and the fluid delivery data. In some aspects, the instruction can include a prescription rate, a prescription volume, or both.

In various aspects, the fluid reservoir 124 comprises a syringe having an internal chamber and a plunger disposed within the internal chamber of the syringe and configured to slide along an inner diameter of the internal chamber when a pressure is applied to the plunger, and actuating the actuator 115 can further include displacing, by the actuator 115, the plunger of the syringe based at least in part on a volume of the syringe and a prescription rate. In some aspects, the motor 121 can be a first motor 121, the actuator 115 can be a first actuator 115, the fluid can be a first fluid, the fluid reservoir 124 can be a first fluid reservoir 124, the tube 130 can be a first tube 130, and the method can further include controlling, by at least one motor driver, a second motor 121 in data communication with the one motor driver based at least in part on the instruction, and actuating, by the second motor 121, a second actuator 115 configured to actuate a second fluid within a second fluid reservoir 124 to deliver a second fluid from the second fluid reservoir 124 to a second tube 130. In some aspects, the first actuator 115 and the second actuator 115 can be actuated sequentially, simultaneously, or individually.

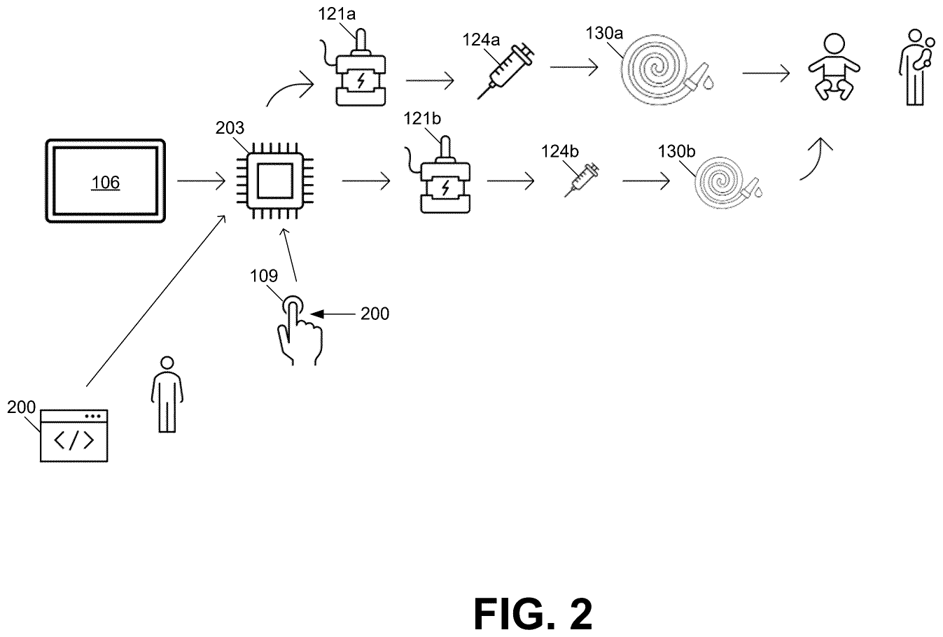

Referring now to FIG. 2, shown is an example process flow of an operation of an enteral feeding pump device 100 according to various embodiments of the present disclosure. First, a user input 200 can be acquired by a computing device 203. In various aspects, a user input 200 can be acquired based at least in part on a user's interactions with one or more user interface 106 elements of a computing device 203 (e.g., buttons 109 of an enteral feeding pump device 100) and/or one or more buttons 109 of an external control device in data communication with the computing device 203. In some aspects, a user input 200 can be acquired based at least in part on a user's interactions with a user interface 106 of the enteral feeding pump 100 and/or via a wireless connection with an external computing device (e.g., a client device). Once a user input 200 is acquired, the computing device 203 can generate an instruction for at least one motor driver based at least in part on the user input 200. Subsequently, one or more motor drivers can control a motor 121 (e.g., 121a, 121b) in data communication with the motor drivers based at least in part on the instruction. The motor 121 can then actuate an actuator 115 configured to actuate a fluid within a fluid reservoir 124 (e.g., 124a, 124b) to deliver the fluid from the fluid reservoir 124 to a tube 130 (e.g., 130a, 130b) connected to a patient. As depicted in FIG. 2, in some examples, one of the fluid reservoirs 124a can hold a larger volume of fluid than the other fluid reservoir 124b. In various aspects, the larger fluid reservoir 124a can be actuated to perform gavage feeding, while the smaller fluid reservoir 124b can be actuated to perform oral feeding.

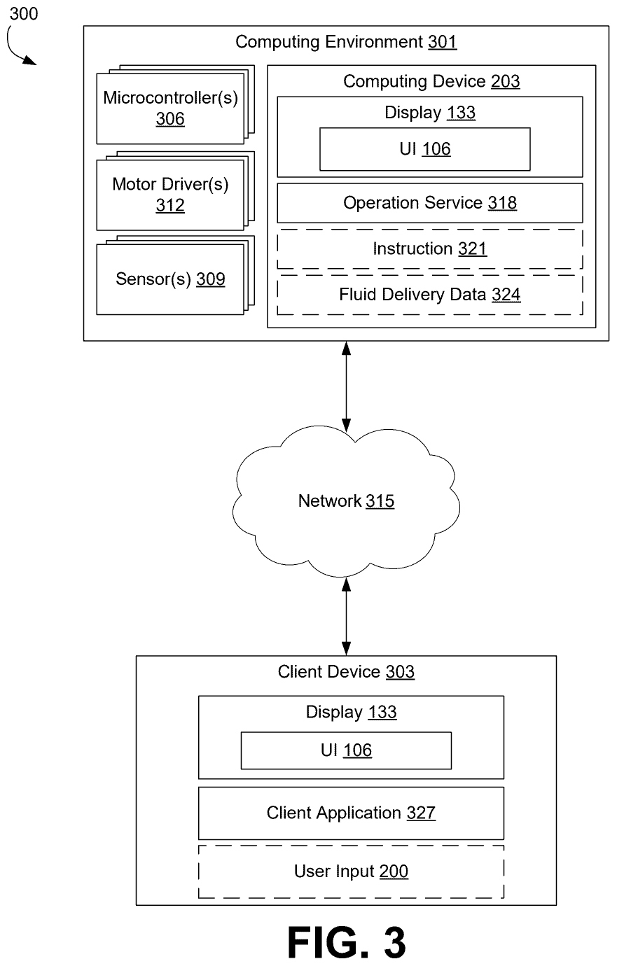

With reference to FIG. 3, shown is a network environment 300 according to various embodiments. The network environment 300 can include a computing environment 301 (including a computing device 203, one or more microcontrollers 306, one or more motor drivers 312, and/or one or more sensors 309, which can be in data communication with each other via wired or wireless connections) and a client device 303 which can be in data communication with each other via a network 315.

The computing device 303 can include one or more computing devices 203 that include a processor, a memory, and/or a network interface. For example, the computing devices 203 can be configured to perform computations on behalf of other computing devices 203 or applications. As another example, such computing devices 203 can host and/or provide content to other computing devices 203 in response to requests for content. In some examples, a computing device 303 can include a microcontroller 306.

The client device 303 can be representative of a plurality of client devices 303 that can be coupled to the network 315. The client device 303 can include a processor-based system such as a computer system. Such a computer system can be embodied in the form of a personal computer (e.g., a desktop computer, a laptop computer, or similar device), a mobile computing device (e.g., personal digital assistants, cellular telephones, smartphones, web pads, tablet computer systems, music players, portable game consoles, electronic book readers, and similar devices), media playback devices (e.g., media streaming devices, BluRay® players, digital video disc (DVD) players, set-top boxes, and similar devices), a videogame console, or other devices with like capability. The client device 303 can include one or more displays 133, such as liquid crystal displays (LCDs), gas plasma-based flat panel displays, organic light emitting diode (OLED) displays, electrophoretic ink (“E-ink”) displays, projectors, or other types of display devices. In some instances, the display 133 can be a component of the client device 303 or can be connected to the client device 303 through a wired or wireless connection.

A user interface 106 can include a network page, an application screen, or other user mechanism for obtaining user input 200. A user interface 106 can be rendered on a display 133 of a client device 303. According to various embodiments of the present disclosure, a user interface 106 can obtain various user inputs 200 for use by the network environment 300. For example, an application screen can be depicted on the display 133 of a client device 303 and can obtain a user input 200 based at least in part on interactions by a user. In some examples, a user interface 106 can include one or more buttons 109 that allow a user to trigger an action within the enteral feeding pump system upon pressing or clicking the buttons 109.

A display 133 is an electronic device, or a component of an electronic device, that presents information visually to a user. In some instances, the display 133 can be a component of the client device 303 or can be connected to the client device 303 through a wired or wireless connection. A display 133 can be used to render a user interface 106 to allow a user to interact with the electronic device. For example, the display 133 of a client device 303 can render a user interface 106 in order to obtain a user input 200 from a user.

A microcontroller 306 can refer to an integrated circuit device designed to perform various control functions within an enteral feeding pump system. A microcontroller 306 can include a central processing unit (CPU), non-volatile memory, volatile memory, and a variety of peripheral interfaces (e.g., timers, analog-to-digital converters, digital-to-analog converters, communication modules (e.g., USB, CAN, UART, etc.), etc.). A microcontroller 306 can be configured to execute a variety of program instructions stored in its memory to monitor and control connected hardware components (e.g., one or more motor drivers 312 and associated motors 121, one or more sensors 309, etc.). In various aspects, one or more computing devices 203 can include a microcontroller 306.

A sensor 309 can refer to a device configured to detect, measure, or monitor a fluid delivery rate (or a prescription rate) and/or a fluid delivery volume. A sensor can monitor various physical conditions or parameters and convert the detected information into a signal that can be interpreted, processed, or transmitted by a computing device 203 (e.g., a microcontroller 306). A sensor can monitor various physical conditions, such as a velocity of a motor 121, a displacement of an actuator 115, a volume of fluid within a fluid reservoir 124, or combinations of any thereof.

A motor driver 312 can refer to an electronic circuit or integrated device configured to control the operation of one or more motors 121 by receiving low-power control signals from a computing device 203 (e.g., a microcontroller 306) and outputting high-power signals suitable for driving the motor 121. A motor driver 312 can function as an intermediary interface between a control logic system and a motor 121, facilitating the amplification, switching, and modulation of electrical power to control motor 121 behavior. A motor driver 312 can be configured to control a direction and/or a speed of a motor 121.

The network 315 can include wide area networks (WANs), local area networks (LANs), personal area networks (PANs), or a combination thereof. These networks can include wired or wireless components or a combination thereof. Wired networks can include Ethernet networks, cable networks, fiber optic networks, and telephone networks such as dial-up, digital subscriber line (DSL), and integrated services digital network (ISDN) networks. Wireless networks can include cellular networks, satellite networks, Institute of Electrical and Electronic Engineers (IEEE) 802.11 wireless networks (i.e., WI-FI®), BLUETOOTH® networks, microwave transmission networks, as well as other networks relying on radio broadcasts. The network 315 can also include a combination of two or more networks 315. Examples of networks 315 can include the Internet, intranets, extranets, virtual private networks (VPNs), and similar networks.

Various applications or other functionality can be executed in the computing device 203. The components executed on the computing device 203 include an operation service 318, and other applications, services, processes, systems, engines, or functionality not discussed in detail herein.

An operation service 318 can be executed to acquire a user input 200. The operation service 318 can be executed to receive a user input 200 from a client device 303, based at least in part on a user's interactions with a user interface 106 on a computing device 203, or based at least in part on a user's interactions with one or more buttons 109 of an enteral feeding pump device 100. Then, the operation service 318 can be executed to generate an instruction 321 for at least one motor driver 312 based at least in part on the user input 200. The operation service 318 can further be executed to acquire fluid delivery data 324 and generate the instruction 321 for at least one motor driver 312 based at least in part on the user input 200 and the fluid delivery data 324.

A user input 200 can represent a variety of data, signals, commands, selections, or interactions provided by a user through a user interface 106 designed to facilitate communication between the user and a computing device 203. A user input 200 can be provided using one or more buttons 109, through a user interface 106 on the display 133 of a client device 303, and/or through a user interface 106 on the display 133 of a computing device 203. A user input 200 can include selections of graphical elements representing certain instructions 321, voice commands, mouse clicks, button 109 clicks, text entries, touchscreen taps or swipes, and/or other interaction modalities that can be processed by the enteral feeding pump system to trigger a corresponding operation, function, or response.

An instruction 321 can refer to a discrete control signal or set of signals generated by a computing device 203 (e.g., a microcontroller 306) in response to a received user input 200. An instruction 321 can be configured to control an operation of a motor 121, including initiating motion of a motor 121, changing a direction of a motor 121, adjusting a speed of a motor 121, or stopping the motor 121. An instruction 321 can include a target fluid volume delivery rate (a target prescription rate) and/or a target fluid volume to be actuated from a fluid reservoir 124 associated with a motor 121. In some aspects, an instruction 321 can be generated based at least in part on fluid delivery data 324 obtained from a sensor 309.

Fluid delivery data 324 can represent a variety of data associated with delivery of a fluid from a fluid reservoir 124 to a patient. Fluid delivery data 324 can include a volume of fluid within a fluid reservoir 124 and/or a volume of fluid being expelled from the fluid reservoir 124 to a patient via a tube 130. Fluid delivery data 324 can also include a fluid delivery rate (or prescription rate) from a fluid reservoir 124 to a patient. For example, fluid delivery data 324 can indicate a fluid is being delivered from a fluid reservoir 124 to a patient at 2 mL/min. In another example, fluid delivery data 324 can indicate a first fluid reservoir 124 has 1 mL of fluid while a second fluid reservoir 124 has 5 mL of fluid. In some examples, fluid delivery data 324 can include an amount of fluid delivered as a result of one or more movements by the actuator 115.

The client device 303 can be configured to execute various applications such as a client application 327 or other applications. The client application 327 can be executed in a client device 303 to access network content served up by the computing device 203 or other servers, thereby rendering a user interface 106 on the display 133. To this end, the client application 327 can include a browser, a dedicated application, or other executable, and the user interface 106 can include a network page, an application screen, or other user mechanism for obtaining user input. The client device 303 can be configured to execute applications beyond the client application 327 such as email applications, social networking applications, word processors, spreadsheets, or other applications.

Next, a general description of the operation of the various components of the network environment 300 is provided. To begin, a user can provide a user input 200 through a user interface 106 of a client device 303 or a user interface 106 of a computing device 203. Then, the operation service 318 can be executed to acquire the user input 200. The operation service 318 can then be executed to generate an instruction 321 for at least one motor driver 312 based at least in part on the user input 200. In some examples, a user input 200 can indicate to start operation of a motor 121 associated with an actuator 115 in mechanical communication with a fluid reservoir 124, and the operation service 318 can be executed to generate an instruction 321 for the motor driver 312 to initiate an action of the motor 121. The operation service 318 can then be executed to send the instruction 321 to one or more motor drivers 312. Subsequently, the motor driver 312 can control a motor 121 in data communication with the motor driver 312 based at least in part on the instruction 321. The motor 121 can then actuate an actuator 115 configured to actuate a fluid within a fluid reservoir 124 to deliver the fluid from the fluid reservoir 124 to a tube 130 connected to a patient.

In some examples, one or more sensors 309 can measure fluid delivery data 324 and provide the fluid delivery data 324 to the operation service 318. The operation service 318 can then be executed to generate an instruction 321 for at least one motor driver 312 based at least in part on the user input 200 and the fluid delivery data 324. For example, a sensor 309 can measure a fluid flow rate of 2 mL/min and a user input 200 can indicate to stop fluid delivery once the patient receives 4 mL of fluid. In this example, the operation service 318 can use the feedback from the fluid delivery data 324 to generate an instruction 321 to stop the motor 121 after two minutes of operation.

In some examples, an instruction 321 can be generated based at least in part on a user input 200 indicating a target prescription rate of 2 mL/min. The motor driver 312 can then control a motor 121 based at least in part on the instruction 321. A sensor 309 can then measure a fluid flow rate of 1 mL/min and send the fluid delivery data 324 to the operation service 318. The operation service 318 can then be executed to generate another instruction 321 based at least in part on the feedback received from the sensor 309 to increase an operation of the motor 121. This example of a closed-loop feedback mechanism can be iteratively repeated until the target prescription rate of 2 mL/min is achieved.

In various examples, the operation service 318 can instead be executed to send a user input 200 to one or more microcontrollers 306. The one or more microcontrollers 306 can then generate an instruction 321 based at least in part on the user input 200 and send the instruction 321 to one or more motor drivers 312. In some examples, the microcontroller 306 can receive fluid delivery data 324 from one or more sensors 309 and generate an instruction 321 based at least in part on the fluid delivery data 324 and the user input 200.

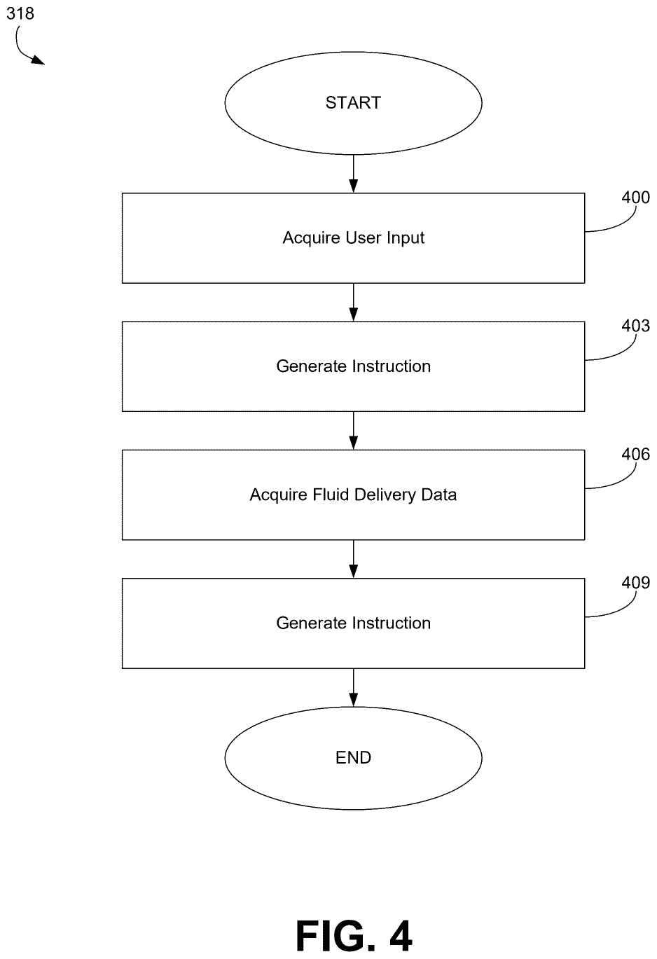

Referring next to FIG. 4, shown is a flowchart that provides one example of the operation of a portion of the operation service 318. The flowchart of FIG. 4 provides merely an example of the many different types of functional arrangements that can be employed to implement the operation of the depicted portion of the operation service 318. As an alternative, the flowchart of FIG. 4 can be viewed as depicting an example of elements of a method implemented within the network environment 300.

At block 400, the operation service 318 can be executed to acquire a user input 200. In various examples, the operation service 318 can be executed to receive an application programming interface (API) call request from a client application 327 that includes a user input 200. In other examples, the operation service 318 can be executed to generate and send an API call request to the client application 327 for a user input 200 and receive the user input 200 in the API call response. In some examples, the operation service 318 can be executed to generate a user input 200 based at least in part on a user's interactions with a user interface 106 rendered on a display 133 of a computing device 203. In further examples, the operation service 318 can be executed to generate a user input 200 based at least in part on a user's interactions with one or more buttons 109 of a computing device 203. For example, a user can press a button 109 on a computing device 203 that provides feedback to the operation service 318 to start an operation of an enteral feeding pump device 100. In various examples, the operation service 318 can instead be executed to send a user input 200 to one or more microcontrollers 306.

Then, at block 403, the operation service 318 can be executed to generate an instruction 321 for at least one motor driver 312 based at least in part on the user input 200. In some examples, a user input 200 can indicate a target prescription rate or target fluid volume to be delivered to a patient. The operation service 318 can be executed to generate an instruction 321 for a motor driver 312 that includes the target prescription rate or target fluid volume. In some examples, a user input 200 can indicate to start an operation of an enteral feeding pump device 100 and the operation service 318 can be executed to generate an instruction 321 for a motor driver 312 to begin operation of a motor 121. The operation service 318 can then be executed to send the instruction 321 to one or more motor drivers 312. In various examples, one or more microcontrollers 306 can instead generate an instruction 321 based at least in part on the user input 200 and send the instruction 321 to one or more motor drivers 312.

Next, at block 406, the operation service 318 can be executed to acquire fluid delivery data 324. The operation service 318 can be executed to receive fluid delivery data 324 from one or more sensors 309 according to various serial communication protocols (e.g., serial peripheral interface (SPI), inter-integrated circuit (I²C), controller area network (CAN), universal serial bus (USB), universal asynchronous receiver/transmitter (UART), etc.). In various examples, a microcontroller 306 can receive fluid delivery data 324 from one or more sensors 309 and the operation service 318 can be executed to receive the fluid delivery data 324 from the microcontroller 306 according to various serial communication protocols.

At block 409, the operation service 318 can be executed to generate an instruction 321 based at least in part on the fluid delivery data 324 and the user input 200. The operation service 318 can be executed to generate an instruction 321 for a motor driver 312 based at least in part on the fluid delivery data 324 and the user input 200. For example, the operation service 318 can receive fluid delivery data 324 indicating 4 mL of fluid has been delivered to a patient. The operation service 318 can then be executed to generate an instruction 321 for a motor driver 312 to stop an operation of a motor 121 based at least in part on a user input 200 indicating a target fluid delivery volume of 4 mL. In some examples, the microcontroller 306 can instead receive fluid delivery data 324 from one or more sensors 309 and generate an instruction 321 based at least in part on the fluid delivery data 324 and the user input 200. After block 409, the process depicted by the flowchart of FIG. 4 can end.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. An enteral feeding pump, comprising:
at least one motor driver;
a fluid delivery system comprising:
at least one motor configured to actuate at least one actuator, wherein the at least one motor is in data communication with the at least one motor driver;
the at least one actuator configured to actuate a fluid within at least one fluid reservoir; and
the at least one fluid reservoir configured to contain the fluid and in fluidic communication with at least one tube, wherein the at least one fluid reservoir is connected to the at least one actuator at a first end of the at least one fluid reservoir and has a receiving end at a second end of the at least one fluid reservoir configured to removably connect to the at least one tube; and
the at least one tube, wherein the at least one tube is in fluidic communication with the at least one fluid reservoir, wherein the at least one tube removably connects at a first end of the at least one tube to the receiving end of the at least one fluid reservoir, and wherein the at least one tube is configured to deliver the fluid from the at least one fluid reservoir when the at least one actuator is actuated by the at least one motor.

2. The enteral feeding pump of claim 1, further comprising a housing configured to enclose the at least one motor driver and the fluid delivery system.

3. The enteral feeding pump of claim 1, wherein the at least one tube comprises a purge port.

4. The enteral feeding pump of claim 1, further comprising:
one or more computing devices comprising a processor and a memory; and
machine-readable instructions that, when executed by the processor, cause the one or more computing devices to at least:
acquire a user input;
generate an instruction for the at least one motor driver based at least in part on the user input; and
send the instruction to the at least one motor driver.

5. The enteral feeding pump of claim 1, wherein the at least one fluid reservoir comprises a syringe having an internal chamber and a plunger disposed within the internal chamber of the syringe and configured to slide along an inner diameter of the internal chamber when a pressure is applied to the plunger by the at least one actuator.

6. The enteral feeding pump of claim 4, further comprising a user interface disposed within an outer surface of the enteral feeding pump and in data communication with the one or more computing devices.

7. The enteral feeding pump of claim 6, wherein the user interface is configured to receive a user input and wherein the at least one motor driver controls the at least one motor based at least in part on the user input.

8. An enteral feeding pump system, comprising:
a housing, wherein the housing comprises:
two or more openings extending from an interior of the housing to an outer surface of the housing, wherein each of the two or more openings is configured to fit a tube;
a control system disposed within the interior of the housing, wherein the control system comprises:
two or more motor drivers;
a computing device comprising a processor and a memory; and
machine-readable instructions that, when executed by the processor, cause the computing device to at least:
acquire a user input;
generate an instruction for at least one of the two or more motor drivers based at least in part on the user input; and
control at least one of the two or more motor drivers based at least in part on the instruction;
a user interface disposed within the outer surface of the housing and in data communication with the control system;
a fluid delivery system disposed within the interior of the housing, wherein the fluid delivery system comprises two or more bays, wherein each of the two or more bays comprises:
a motor configured to actuate an actuator, wherein the motor is in data communication with at least one motor driver;
the actuator configured to actuate a fluid within a fluid reservoir; and
the fluid reservoir configured to contain the fluid and in fluidic communication with the tube, wherein the fluid reservoir is connected to the actuator at a first end of the fluid reservoir and has a receiving end at a second end of the fluid reservoir configured to removably connect to the tube; and
two or more tubes, wherein each of the two or more tubes is in fluidic communication with the fluid reservoir, wherein each of the two or more tubes removably connects at a first end to the receiving end of the fluid reservoir, and wherein each of the two or more tubes is configured to deliver the fluid from the fluid reservoir when the actuator is actuated by the motor.

9. The system of claim 8, wherein the user interface comprises one or more buttons, and wherein the machine-readable instructions that, when executed by the processor, cause the computing device to generate the instruction further cause the computing device to generate the instruction based at least in part on an activation of the one or more buttons.

10. The system of claim 8, wherein the tube comprises a purge port.

11. The system of claim 8, wherein the machine-readable instructions that, when executed by the processor, cause the computing device to acquire the user input further cause the computing device to at least:
acquire fluid delivery data; and
generate the instruction for at least one of the two or more motor drivers based at least in part on the user input and the fluid delivery data.

12. The system of claim 8, wherein the at least one fluid reservoir comprises a syringe having an internal chamber and a plunger disposed within the internal chamber of the syringe and configured to slide along an inner diameter of the internal chamber when a pressure is applied to the plunger by the at least one actuator.

13. The system of claim 8, further comprising a first fluid reservoir and a second fluid reservoir, wherein a volume of the first fluid reservoir is less than a volume of the second fluid reservoir.

14. The system of claim 12, wherein each of the two or more bays further comprise a clamp assembly configured to receive a flange of the syringe.

15. A method of operating an enteral feeding pump, comprising:
acquiring a user input;
generating an instruction for at least one motor driver based at least in part on the user input;
controlling, by the at least one motor driver, a motor in data communication with the at least one motor driver based at least in part on the instruction; and
actuating, by the motor, an actuator configured to actuate a fluid within a fluid reservoir to deliver the fluid from the fluid reservoir to a tube.

16. The method of claim 15, wherein acquiring the user input further comprises:
acquiring fluid delivery data; and
generating the instruction for at least one motor driver based at least in part on the user input and the fluid delivery data.

17. The method of claim 15, wherein the instruction comprises a prescription rate, a prescription volume, or both.

18. The method of claim 15, wherein the fluid reservoir comprises a syringe having an internal chamber and a plunger disposed within the internal chamber of the syringe and configured to slide along an inner diameter of the internal chamber when a pressure is applied to the plunger, and wherein actuating the actuator further comprises displacing, by the actuator, the plunger of the syringe based at least in part on a volume of the syringe and a prescription rate.

19. The method of claim 15, wherein the motor is a first motor, the actuator is a first actuator, the fluid is a first fluid, the fluid reservoir is a first fluid reservoir, the tube is a first tube, and the method further comprises:
controlling, by the at least one motor driver, a second motor in data communication with the at least one motor driver based at least in part on the instruction; and
actuating, by the second motor, a second actuator configured to actuate a second fluid within a second fluid reservoir to deliver a second fluid from the second fluid reservoir to a second tube.

20. The method of claim 19, wherein the first actuator and the second actuator are actuated sequentially, simultaneously, or individually.
